(19) United States
(12) Patent Application Publication
(10) Pub. No.: US 2026/0060751 A1
(43) Pub. Date: Mar. 5, 2026

(54) METHOD FOR MODELLING AN ENDOPROTHESIS IMPLANTED IN A PORTION OF A PATIENT AORTA

(71) Applicant: PREDISURGE, Saint-Étienne (FR)

(72) Inventors: Joris VERMUNT, Saint-Etienne (FR); Jean-Noël ALBERTINI, Saint-Etienne (FR); David PERRIN, Saint-Etienne (FR)

(73) Assignee: PREDISURGE, Saint-Étienne (FR)

(21) Appl. No.: 19/105,856

(22) PCT Filed: Sep. 8, 2023
(86) PCT No.: PCT/EP2023/074695

§ 371 Date: Feb. 24, 2025

Related U.S. Application Data
(60) Provisional application No. 63/404,700, filed on Sep. 8, 2022.

(30) Foreign Application Priority Data
Mar. 15, 2023 (EP) 23162028.7

(51) Int. Cl. A61B 34/10; G16H 30/40

(52) U.S. Cl. CPC A61B 34/10; G16H 30/40; A61B 2034/104; A61B 2034/105; A61B 2034/107

(57) ABSTRACT

A computer-implemented method for simulating an interaction between an endoprosthesis dedicated to be implanted in a portion of a patient aorta and said portion of the patient aorta, so as to determine areas with risk of surgical complications.

### FIELD

The present invention relates to the field of numerical simulations of cardiovascular surgical interventions.

More specifically, the invention concerns a computer implementing method for modelling an endoprosthesis implanted in a portion of a patient aorta in order to improve surgery outcomes.

### BACKGROUND

Cardiovascular surgical interventions are often followed by complications such as endoleaks, endoprosthesis migration, endoprosthesis infolding, endoprosthesis occlusion, thrombosis, and endoprosthesis kinks. In this context, there is room for improvement notably in the pre-operative phase of planification. The present invention provides a solution to allow evaluation of outcomes of the surgery beforehand and also personalization of the surgery.

### SUMMARY

This invention thus relates to a computer-implemented method for simulating an interaction between an endoprosthesis dedicated to be implanted in a portion of a patient aorta and said portion of said patient aorta, so as to determine areas with risk of surgical complications, comprising the steps of:
receiving 3D images of said portion of the patient aorta,
reconstructing a deformable tridimensional digital model of said portion of the patient aorta based on said 3D images, the reconstructed deformable tridimensional digital model being referred to as aorta model,
positioning a tridimensional digital model of said endoprosthesis at a planned location in the aorta model, said tridimensional model of said endoprosthesis and said aorta model forming together an implantation model,
simulating said interaction based on a computation using said implantation model,
determining, in the implantation model, based on results of said simulated interaction, at least one risk index of surgical complications for predefined areas.

For instance, one global risk index can be determined, for the whole endoprosthesis. In other examples, the predefined areas depend on the type of surgical complications. For instance, when the surgical complications is a type Ia endoleak, the defined areas are the proximal covered stents. In the present disclosure, by “endoprosthesis”, it is meant a “stent-graft”. Covered stent components of a stent-graft are used to secure the upper or beginning portion of the stent graft within the blood vessel. These components help to create a sealed and stable connection between the stent graft and the native vessel, preventing blood from leaking into or around the aneurysm or damaged area. In the present disclosure, the term “proximal” means upstream of the blood flow; for example proximal stents are the stents at the top of the stent-graft that will be deployed closer to the heart. When the surgical complication is a risk of migration, the defined area is the proximal uncovered stent. When the surgical complication is a risk of infolding or occlusion, the predefined area is the whole endoprosthesis. The predefined areas depend on the endoprosthesis manufacturer to be precise.

Thus, the method allows, by using patient-related data such as anatomical images and planification data such as a numerical model of an endoprosthesis planned to be implanted in the body of a patient, foreseeing potential areas of surgical complications. Meanwhile, the method allows personalizing a surgical intervention comprising an endoprosthesis implantation.

Advantageously, the surgical complications comprise at least one among endoleaks, endoprosthesis migration, endoprosthesis infolding, endoprosthesis occlusion, thrombosis, and endoprosthesis kinks.

In some embodiments, the computation involving said implantation model comprises a computation of a geometrical deformation of said tridimensional digital model of said endoprosthesis inside the aorta model.

In some embodiments, the computation of the geometrical deformation is performed by finite element analysis.

Advantageously, the 3D images are preoperative CT scan images. Thus, the method allows foreseeing surgical complications before a surgical intervention is carried out. This is advantageous, as current techniques are based on post-operative data. Therefore, the method allows improving the planification of a surgical intervention by rendering it possible testing different equipment to be used during the surgical intervention.

In some embodiments, the interaction is an endovascular repair of aortic aneurysms.

In some embodiments, the method further comprises, after determining the areas with risk of surgical complication, a step of determining, for each area with risk of surgical complication, a risk level.

In some embodiments, the step of determining a risk level comprises a computation involving an apposition index or an aspect ratio.

In some embodiments, each risk level is associated with a corresponding display pattern, the method further comprising a step of applying the corresponding display pattern to each area with risk of surgical complication.

Advantageously, the method further comprises a step of displaying the implantation model on a visualization unit with the areas with risk of surgical complication with the applied corresponding display pattern.

Another aspect of the invention relates to a method for planning an abdominal aortic aneurysm repair surgery using areas with risk of surgical complications determined by the method according to any of the preceding claims. As already mentioned, by using the method for simulating an interaction between an endoprosthesis and a portion of a patient aorta, it is possible to test different models of endoprosthesis and to select one that will minimize the risk of surgical complications.

Another aspect of the invention relates to a device comprising a processor configured to carry out the method previously described.

According to one embodiment, the device for simulating an interaction between an endoprosthesis dedicated to be implanted in a portion of a patient aorta and said portion of said patient aorta, so as to determine areas with risk of surgical complications, said device comprises:
at least one input configured to receive 3D images of said portion of the patient aorta,
at least one processor configured to:
   reconstruct a deformable tridimensional digital model of said portion of the patient aorta based on said 3D images, the reconstructed deformable tridimensional digital model being referred to as aorta model,
   position a tridimensional digital model of said endoprosthesis at a planned location in the aorta model, said tridimensional model of said endoprosthesis and said aorta model forming together an implantation model,
   simulate said interaction based on a computation using said implantation model,
   determining, in the implantation model, based on results of said simulated interaction, at least one risk index of surgical complications for predefined areas;
at least one output configured to provide said at least one risk index of surgical complications for predefined areas.

Another aspect of the invention relates to a computer program product comprising instructions which, when the program is executed by a computer, cause the computer to carry out the method previously described.

Another aspect of the invention relates to a surgical method for repairing an abdominal aortic aneurysm of a subject comprising a preliminary step of performing the method previously described.

The present disclosure further pertains to a non-transitory program storage device, readable by a computer, tangibly embodying a program of instructions executable by the computer to perform a method for simulating, compliant with the present disclosure.

Such a non-transitory program storage device can be, without limitation, an electronic, magnetic, optical, electromagnetic, infrared, or semiconductor device, or any suitable combination of the foregoing. It is to be appreciated that the following, while providing more specific examples, is merely an illustrative and not exhaustive listing as readily appreciated by one of ordinary skill in the art: a portable computer diskette, a hard disk, a ROM, an EPROM (Erasable Programmable ROM) or a Flash memory, a portable CD-ROM (Compact-Disc ROM).

### Definitions

In the present invention, the following terms have the following meanings:

The terms “adapted” and “configured” are used in the present disclosure as broadly encompassing initial configuration, later adaptation or complementation of the present device, or any combination thereof alike, whether effected through material or software means (including firmware).

The term “processor” should not be construed to be restricted to hardware capable of executing software, and refers in a general way to a processing device, which can for example include a computer, a microprocessor, an integrated circuit, or a programmable logic device (PLD). The processor may also encompass one or more Graphics Processing Units (GPU), whether exploited for computer graphics and image processing or other functions. Additionally, the instructions and/or data enabling to perform associated and/or resulting functionalities may be stored on any processor-readable medium such as, e.g., an integrated circuit, a hard disk, a CD (Compact Disc), an optical disc such as a DVD (Digital Versatile Disc), a RAM (Random-Access Memory) or a ROM (Read-Only Memory). Instructions may be notably stored in hardware, software, firmware or in any combination thereof.

“Machine learning (ML)” designates in a traditional way computer algorithms improving automatically through experience, on the ground of training data enabling to adjust parameters of computer models through gap reductions between expected outputs extracted from the training data and evaluated outputs computed by the computer models.

### BRIEF DESCRIPTION OF THE DRAWINGS

FIG. 1 illustrates a device for simulating an interaction between an endoprosthesis dedicated to be implanted in a portion of a patient aorta.

FIG. 2 is an example of a flow-chart representing a method for simulating an interaction between an endoprosthesis dedicated to be implanted in a portion of a patient aorta.

FIGS. 3a and 3b respectively illustrate a cut plane and a plurality of bidimensional cross-sections in an in-use implementation model.

FIG. 4 illustrates a prepared manifest obtained from an original manifest.

FIG. 5 is an example of a flow-chart representing a method for computing geometrical parameters used to determine at least one risk index.

FIG. 6 shows two curves representing respectively the mean aorta radius and the mean graft radius along a portion of the centerline of the aorta of a patient.

FIG. 7 is an example of an in-use implementation model where risk indices related to an oversizing of a deployed endoprosthesis are graphically represented.

FIG. 8 is an example of an in-use implementation model where risk levels related to an apposition quality (i.e., apposition index) of a deployed endoprosthesis are graphically represented.

FIG. 9 is an example of an in-use implementation model where risk levels related to an anchorage quality of a deployed endoprosthesis are graphically represented.

FIGS. 10a and 10b respectively illustrate an in-use implementation model corresponding to a first endoprosthesis model and to a second endoprosthesis model, where a risk of endoleak is graphically represented.

### DETAILED DESCRIPTION

This invention relates to a computer-implemented method 100 for simulating an interaction between an endoprosthesis dedicated to be implanted in a portion of a patient aorta and said portion of said patient aorta, so as to determine areas with risk of surgical complications.

Examples of surgical complications are endoleaks, endoprosthesis migration, endoprosthesis infolding, endoprosthesis occlusion, thrombosis, endoprosthesis kinks.

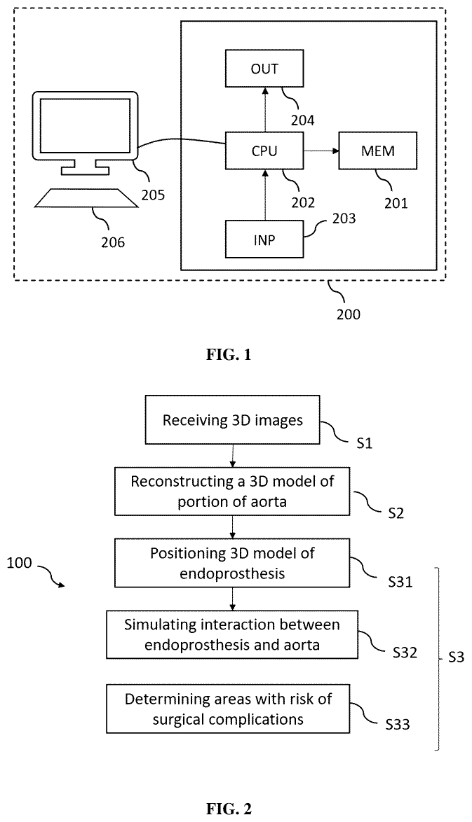

The method 100 may be implemented with a device 200 such as illustrated on FIG. 1.

FIG. 1 illustrates a device 200 for simulating an interaction between an endoprosthesis dedicated to be implanted in a portion of a patient aorta and said portion of said patient aorta, so as to determine areas with risk of surgical complications according to embodiments of the invention.

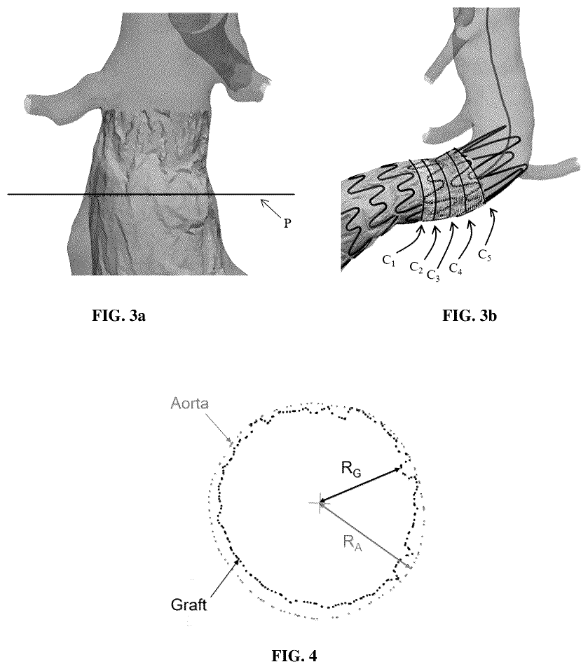

In these embodiments, the device 200 comprises a computer, this computer comprising a memory 201 to store program instructions loadable into a circuit and adapted to cause a circuit 202 to carry out steps of the methods of FIGS. 2 and 3 when the program instructions are run by the circuit 202. The memory 201 may also store data and useful information for carrying steps of the present invention as described above.

### The Circuit 202 May be for Instance:

a processor or a processing unit adapted to interpret instructions in a computer language, the processor or the processing unit may comprise, may be associated with or be attached to a memory comprising the instructions, or
the association of a processor/processing unit and a memory, the processor or the processing unit adapted to interpret instructions in a computer language, the memory comprising said instructions, or
an electronic card wherein the steps of the invention are described within silicon, or
a programmable electronic chip such as a FPGA chip (for «Field-Programmable Gate Array»).

The computer may also comprise an input interface 203 for the reception of input data and an output interface 204 to provide output data. Examples of input data and output data will be provided further.

To ease the interaction with the computer, a screen 205 and a keyboard 206 may be provided and connected to the computer circuit 202.

FIG. 2 is a flowchart illustrating an example of steps to be executed to implement the computer-implemented method 100. It is supposed that an endoprosthesis will be implanted in a portion of an aorta of a patient during an intervention and that, the method 100 will determine potential risks of surgical complications based on a numerical simulation of the implantation of the endoprosthesis in the portion of the patient's aorta.

The method 100 is typically implemented ahead of time of the intervention. When the endoprosthesis will be implanted in the portion of the patient's aorta, the endoprosthesis will interact with the portion of the patient's aorta. The method 100 comprises a step of a numerical simulation of this interaction, as will be described below.

In a step S1, 3D images of a portion of a patient aorta are received. For instance, the 3D images are preoperative CT scan images. The 3D images may be input through the input interface 203 of the device 200.

In a step S2, the 3D images are used to reconstruct a deformable tridimensional digital model of the portion of the patient aorta. For instance, the reconstruction is performed by the circuit 202 of the device 200. The reconstructed deformable tridimensional digital model is referred to as aorta model. For instance, when used in a numerical simulation, a technique such as described in the international patent application WO 2018/073505 A1 may be used to simulate the deformation of the aorta model.

In a step S3, a numerical simulation of the implantation of the endoprosthesis in the portion of the patient's aorta is carried out, as follows.

In a first substep S31, a tridimensional digital model of endoprosthesis is positioned at a planned location in the aorta model. The tridimensional digital model of the endoprosthesis and the aorta model form together an implantation model. For instance, the planned location corresponds to a location determined by a health practitioner responsible for the intervention. This step of positioning is performed by registering the tridimensional digital model of said endoprosthesis, preferably the proximal extremity of the tridimensional digital model of said endoprosthesis, at the planned location in the aorta model.

In a second substep S32, the interaction between the endoprosthesis and the portion of the patient's aorta is simulated using the implantation model.

In a third substep S33, areas of the portion of the patient's aorta, areas with risk of surgical complications are determined based on results of said simulated interaction.

The substeps S31, S32 and S33 of step S3 may be implemented by the circuit 202 of the device 200.

According to some embodiments, in the second substep S32, the interaction is simulated using a finite element analysis method by computing contact forces between the endoprosthesis and the aorta model, then applying the computed contact forces to the tridimensional digital model of the endoprosthesis, so as to generate a deployed in-use representation of the endoprosthesis. Meanwhile, the aorta model is deformed accordingly. The generated deployed in-use representation of the endoprosthesis is referred to as deployed model. The deployed model and the deformed aorta model form together a so-called in-use implantation model.

In some embodiments, the third substep S33 may involve computing at least one risk index. For example, a global risk index is computed. In another example, a map of risk indices, where each risk index corresponds to a position in the in-use implantation model, is computed. The at least one risk index may be computed based on indicators computed from statistical data from geometrical parameters.

In some embodiments, the geometrical parameters are computed as follows. Bidimensional cross-sections (or slices) of the in-use implantation model are computed.

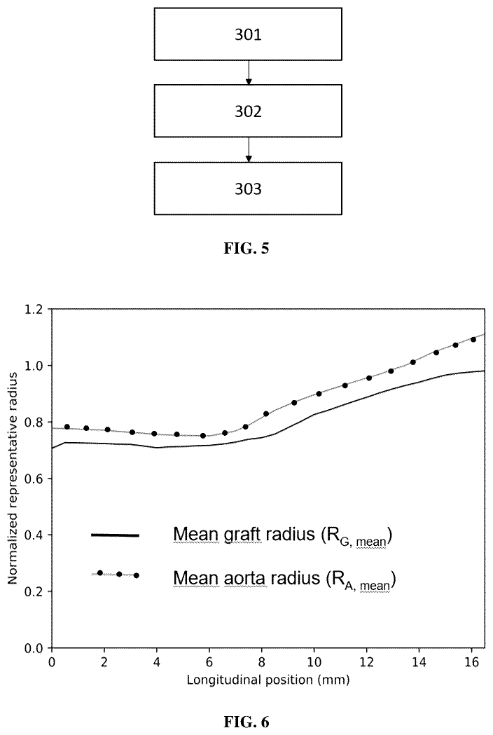

A cut plane corresponds to each cross-section. Such a cut plane P is illustrated on FIG. 3a. For instance, the deformed aorta model comprises a centerline. In this case, the bidimensional cross-sections can be computed along the centerline, as illustrated on FIG. 3b with the curves C₁, C₂, C₃, C₄ and C₅. Those curves represent intersections of cut planes with the in-use implantation model at different points the centerline of the deformed aorta model. For instance, the deployed model comprises a stent portion and a graft portion. In a given bidimensional cross-section, a first set of points corresponds to the section of the aorta model in the corresponding cut plane, a second set of points corresponds to the section of the graft portion and a third set of points corresponds to the section of the stent portion in the corresponding cut plane. The distance of a point of the first set to the corresponding point on the centerline is denoted RAi, the distance of a point of the second set to the corresponding point on the centerline is denoted RGj and the distance of a point of the third set to the corresponding point on the centerline is denoted as RSk. FIG. 4 illustrates how the distances RAi and RGj are computed. The initial diameter of the tridimensional digital model of the endoprosthesis (not deployed) is denoted as RG0. Geometrical parameters can then be computed from the bidimensional cross-sections by implementing a computation procedure 300. An example of the computation procedure 300 is illustrated on FIG. 5 and will be described below. The computation procedure 300 can for instance be implemented by the circuit 202 of the device 200, and data used during the computation procedure 300 may be stored in the memory 201 of the device 200.

In a step 301, the list of the distances RAi, RGj and RSk are stored. For instance, the list is stored in the memory 201 of the device 200.

In a step 302, the distances RAi, RGj and RSk are normalized by division by RG0, so as to obtain a list of so-called normalized aorta radii RAnormi, a list of so-called normalized graft radii RGnormj and a list of so-called normalized stent radii RSnormk.

In a step 303, statistical data are computed from the lists stored and/or obtain at steps 301 and 302. The statistical data may comprise mean, maximum, minimum or standard deviation values.

After the computation procedure 300, a list of indicators is computed, that will be used in the computation of the at least one risk index.

For instance, a list of indicators I1 to I14 that can be derived from the data stored and determined at steps 301 and 302 may be as follows:
I1: apposition between the aorta and the endoprosthesis;
I2: aorta, stents and graft conicity;
I3: aorta, stents and graft straightness;
I4: aorta, stents and graft circularity;
I5: aorta, stents and graft diameter variability;
I6: aorta, stents and graft stress;
I7: pressure of the endoprosthesis on the aorta;
I8: aorta and graft cross-section area;
I9: aorta and graft roughness;
I10: aorta and endoprosthesis angulation;
I11: stents oversize;
I12: surface of calcifications;
I13: surface of thrombus;
I14: patient characteristics (age, medical history).
   For instance, the indicators can be computed from the data stored at step 301 and/or obtained at step 302 by the circuit 202 of the device 200.

In the present disclosure, the term “apposition” designates the fitting (for example, alignment, positioning or placement) of the endoprosthesis within the aorta, and more specifically the contact or diameter difference between the aorta and the graft or between the aorta and the stents.

The at least one risk index may be computed from the indicators by different methods.

For instance, the at least one risk index can be computed by means of a linear or quadratic discriminant analysis. Such an analysis is a statistical analysis that classifies a data set into predefined groups. A scatter plot represents each group in an n-dimensional space, where n is the total number of indicators. The distance between each point and the barycenter of each group is used to predict whether a case belongs to a group. Decision surfaces are computed to delimit the different groups. These surfaces can be more or less complex (linear, quadratic, . . . , etc). This method is similar to learning techniques. Different decision surfaces in an n-dimensional space are defined during the learning process on known cases, which allows then to calculate the probability of belonging to one group or the other for a new case.

In another example, the at least one risk index can be computed by a machine learning classification algorithm. The machine learning classification algorithm may be trained to output at least one risk index based on the classification of an unclassified data set into predefined groups. The machine learning classification algorithm may be trained with a training set of indicators. An example of machine learning classification algorithm is a support vector machine. In this algorithm, the predefined groups are defined over a parameter space. To delimit the groups, trained data sets are discriminated in the parameter space. To help this discrimination, a kernel function is applied on the training data sets. This kernel function can be a linear, sigmoid or radial basis function. A boundary is calculated in the parameter space to separate the different groups. The positioning of a new data set in this space gives a classification in a predefined group.

In yet another example, the at least one risk index can be computed by means of weighted averages. For instance, the at least one risk of index can be computed as a statistical value of several weighted averages of the indicators.

A simplified version of this latter weighted averages method is the computation of a unique weighted average of all indicators.

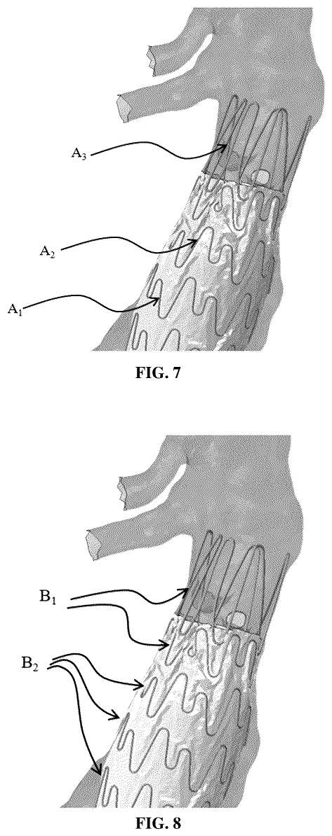

In a typical example where the surgical complication to be determined by the method 100 is a type Ia endoleak, a relevant indicator is the indicator I1 (also referred to as apposition index), i.e. the apposition between the aorta and the endoprosthesis. The apposition can be computed by different methods. In a first method, the sum of the difference between the graft and aorta radii can be computed. FIG. 6 shows two curves representing the mean aorta radius and the mean graft radius along a portion of the centerline of the aorta. Here, the mean is calculated among all values in the cross section corresponding to a point on the centerline of the aorta. In a second method, the apposition is computed as the longest area with a lack of apposition. In a third method, the apposition is computed as the length of apposition higher than a given criterion.

In a second example an alternative relevant indicator to be calculated is an aspect ratio. Said aspect ratio may be calculated as the ratio of the difference between the maximal and minimal aortic normalized radii, to the difference between the maximal and minimal graft normalized radii after deployment simulation.

In some embodiments, the results of the method 100 for simulating an interaction between an endoprosthesis dedicated to be implanted in a portion of a patient aorta and said portion of said patient aorta, so as to determine areas with risk of surgical complications, may be received, gathered and stored by a Web platform. The Web platform may allow visualizing those results. For instance, the Web platform allows visualizing the deployed model and the deformed aorta model, forming together a so-called in-use implantation model, with the areas with risk of surgical complications with a corresponding display pattern.

According to one embodiment, each risk level (for one area/zone) is associated with a corresponding display pattern. Said display pattern may be a color, for example chosen from a color scale, or a texture pattern, for example comprising dots, stars or any other mark covering the area/zone. The method comprises applying said corresponding display pattern to each area/zone for which the risk of surgical complication had been calculated. Furthermore, the method may be further configured to display the implantation model on a visualization unit wherein the areas for which the risk of surgical complication is calculated are visualized with the corresponding display pattern, as shown in FIGS. 7, 8 and 9.

In some embodiments, the risk indices (i.e., risk levels) are percentages comprised between 0 and 100%, where 100% represents a high risk level. FIG. 7 represents an example of in-use implantation model where areas with different risk indices, are visible. For instance, this in-use model may be visualized on the screen 205 of the device 200. In the visualization example of FIG. 7, the risk indices (i.e., representing risk levels) are related to the oversizing criterion of the deployed endoprosthesis and are determined by computing an oversizing factor, such as the indicator I11. For instance, the area A₁ of the stent, colored in dark blue (i.e., corresponding display pattern), corresponds to an oversizing comprised between 0 and 5%; the area A₂ of the stent, colored in light blue, corresponds to an oversizing comprised between 5 and 10%; the area A₃ of the stent, colored in yellow, corresponds to an oversizing comprised between 20 and 25%.

In one embodiment, the determination of the risk level for one area comprises the computation of the apposition index and/or an aspect ratio. In some embodiments, a risk level is attributed to areas of the in-use implantation model depending on the value of the risk index associated with the areas. For instance, when the risk index associated with an area is less than a first threshold value, the area is assigned a low risk level. When the risk index associated with an area is comprised between the first threshold value and a second threshold value, the area is assigned a medium risk level. When the risk index associated with an area is higher than the second threshold value, the area is assigned a high risk level.

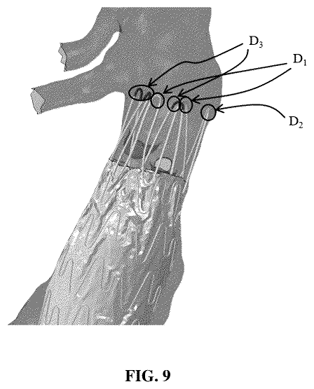

In some embodiments, the risk indices can be related to a specific characteristic pertaining to the interaction between the endoprosthesis and the portion of the patient's aorta. For instance, the risk indices can pertain to the quality of apposition of the endoprosthesis in the portion of the patient's aorta. FIG. 8 illustrates an example of in-use implantation model where the level quality of apposition of several areas is can be visualized. For instance, this in-use model may be visualized on the screen 205 of the device 200. For instance, in the area/zone B₁ of the stent, colored in green (i.e., corresponding display pattern), the apposition of the endoprosthesis is good. In the area B₂, colored in red, there is a lack of apposition. In another example, the risk indices can pertain to the quality of anchorage of the endoprosthesis in the portion of the patient's aorta. FIG. 9 illustrates an example of in-use implantation model where the level of quality of anchorage of the endoprosthesis at several areas can be visualized. For instance, this in-use model may be visualized on the screen 205 of the device 200. For instance, the areas/zone of the stent referred as D₁, colored in green (i.e., corresponding display pattern), present a good anchorage. The area referred as D₂, colored in yellow, presents a low anchorage. In the areas referred as D₃, colored in red, there is no anchorage.

### EXAMPLES

The present invention is further illustrated by the following examples.

### Example 1: Prediction of Type Ia Endoleaks after Endovascular Aneurysm Repair (EVAR)

In this example, a retrospective cohort study was performed. The study population consisted of all patients who underwent implantation of an Endurant® AAA Endovascular Graft (Medtronic, Dublin, Ireland) and presented an early or late (maximum after 5 years) type Ia endoleaks between January 2012 and September 2017 at two high-volume aortic centres. Control cases consisted of all patients who underwent implantation of the same graft without early or late type Ia endoleaks during the same period and a minimum follow-up of 5 years.

Pre-operative CT-scan were analysed with a dedicated workstation providing centerline extraction. AAA maximal diameter and proximal neck characteristics were measured using 3D and centerline reconstructions. Hostile neck anatomy was defined as one or more of the following: (1) neck length≤15 mm, defined as the length over which the aortic diameter remains within 10% of the infra renal diameter (2) conical or tapered neck using the “neck coefficient” introducing by Albertini et al. 1, (3) circumferential neck thrombus or major calcifications, (4) supra renal angulation ≥45 degrees and (5) infra renal angulation ≥60 degrees. Inside Endurant II/IIs's Instructions For Use (IFU) were defined with the following neck characteristics: (1) proximal neck length of ≥10 mm, defined as the length over which the aortic diameter remains within 10% of the infra renal diameter (2) infra renal angulation of <60°; (3) aortic neck diameters with range of 19 to 32 mm. All patients were then classified for hostile and outside the IFU criteria.

Computational technology was based on finite-element analysis and assessed the deformations induced by the device-host interaction resulting in a prediction of graft behaviour and arterial displacement. This work was based on proprietary algorithms of the applicant developed to simulate deployment of endovascular devices in patient-specific aortic aneurysms models. As the purpose of the study focused on EVAR proximal fixation, only the main graft deployment was simulated in our model.

High resolution CT-scans were used to generate patient-specific 3D models of the aorta, from the supra-renal aorta to the extern iliac arteries, including the renal arteries (aortic segmentation step) and assigned an orthotropic elastic behaviour. As calcifications of the aortic wall and intra-luminal thrombus were not modelled, the aortic wall, iliac arteries and the renal arteries were considered as homogeneous surface with a constant surface of 1.5 mm, 1 mm and 1 mm respectively. The main EndurantII/IIs® device without extension was digitally reproduced on the basis of real characteristics' design of the endograft deployed in the corresponding patient. Mechanical properties of stentgraft fabric and stent rings were obtained from literature or from house mechanical testing of samples obtain from manufacturer. For the stent rings, a super-elastic material behaviour was used and the fabric was modelled as an orthotropic elastic material. Oversizing of the stent rings was respected. Deployment of the personalized stentgraft in its corresponding model of the aorta was performed using the commercially available Abaqus/Explicit 2018 finite element solver (Dassault Systèmes, Paris, France). Level of deployment was achieved in an idealized situation, just below the lowest renal artery, and blinded to the real per-operative EVAR deployment. Simulation technology assessed the deformations induced by the device-host interaction resulting in a prediction of stentgraft behaviour and arterial displacement. The methodology implied a step called “morphing”, which is presented in the method described in the international patent application WO 2018/073505 A1, during which the aortic surface was numerically deformed until a cylindric shape is obtained, allowing deployment of a stentgraft inside. Then a reverse deformation of the aortic aorta was achieved while maintaining the stentgraft in place.

Mean oversizing and stentgraft apposition assessment on the two first covered stents of the main stentgraft were measured on simulation results.

Aortic and graft sections were made every 0.5 mm at the level of the first two stents of the covered part of the stentgraft. For each section, a normalized graft and aorta representative radii were calculated in order to avoid systematic bias relative to diameter.

“Aspect ratio” was evaluated as the ratio of the difference between the maximal and minimal aortic normalized radii, to the difference between the maximal and minimal graft normalized radii after deployment simulation.

Apposition was evaluated by the difference of the area under the curve of the aortic representative radii and the area under the curve of the graft representative radii.

A risk index was calculated by the method 100 previously described, based on the aspect ratio indicator and on the apposition factor. A quadratic discriminant analysis has been used to classify the cases in two groups: with or without risk of type Ia endoleak.

Normality could not be assumed for a large part of data because of small numbers. Thus, descriptive data were displayed as medians with interquartile ranges (Q1, Q3). Statistical differences in indexes were assessed with the non-parametric Mann Whitney U test for continuous variables and χ² or the Fischer's exact test for categorical variables. Statistical significance was assumed at p<0.05; all reported p-values were two-sided. All statistical analysis were performed with the R statistical software version 4.2.2 (A language and environment for statistical computing. R foundation for Statistical Computing, Vienna, Austria).

### Results

162 cases were analysed and 15 type Ia endoleaks cases and 14 control cases with sufficient follow-up were identified. Mean follow up was 1.9 years [0-8.7] in the type Ia endoleaks group and 6.8 years [5.2-10.3] in the control group. In the type Ia endoleak group, 8 cases (53.3%) presented a per-operative or early type Ia endoleak and 7 (46.7%) late type Ia endoleaks (from 8 months to 5 years). Table 2 shows, for the 15 type Ia endoleaks cases and 14 control cases, the comparison between the real observation of type Ia endoleaks and the prediction with the method 100. The filled circles represent a presence of a type Ia endoleak, while the crosses represent an absence of a type Ia endoleak.

| | TYPE Ia ENDOLEAK | | Endoleak | |
|---|---|---|---|---|
| Case | | Simulation with the | delay/Last | |
| number | Real Observation | method 100 | follow-up | |
| 1 | ● | ● | Peroperative | |
| 2 | ● | ● | 5 | years |
| 3 | ● | ● | Peroperative | |
| 4 | ● | ● | Peroperative | |
| 5 | ● | ● | Peroperative | |
| 6 | ● | ● | Peroperative | |
| 7 | ● | ● | 1 | day |
| 8 | ● | ● | 5 | years |
| 9 | ● | ● | Peroperative | |
| 10 | ● | ● | Peroperative | |
| 11 | ● | ● | 2 | years |
| 12 | ● | ● | 5 | years |
| 13 | ● | ● | 3 | years |
| 14 | ● | ● | 8 | months |
| 15 | ● | ● | 2 | years |
| 16 | X | X | 6 | years |
| 17 | X | X | 7 | years |
| 18 | X | X | 9 | years |
| 19 | X | X | 5 | years |
| 20 | X | X | 7 | years |
| 21 | X | X | 7 | years |
| 22 | X | X | 5 | years |
| 23 | X | X | 5 | years |
| 24 | X | X | 5 | years |
| 25 | X | X | 10 | years |
| 26 | X | X | 8 | years |
| 27 | X | X | 8 | years |
| 28 | X | X | 9 | years |
| 29 | X | X | 7 | years |

### Example 2: Assistance in the Choice of Endoprosthesis During the Planification Stage

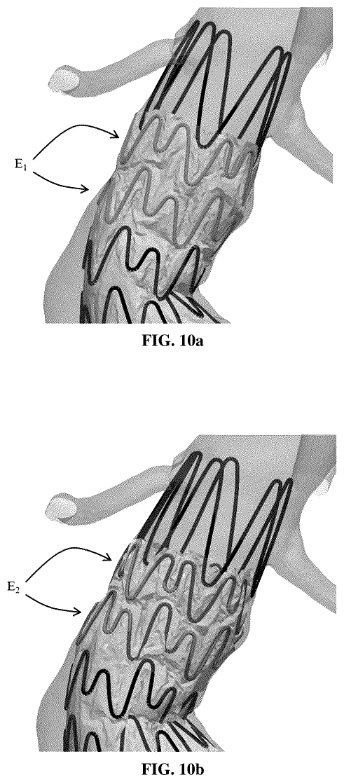

By determining areas with risk of surgical complications, the method 100 can be of help to choose a suitable endoprosthesis that will minimize the risk of surgical complication. FIG. 10a represents an example of in-use implantation model where a model of an Endurant II device of type ETBF2516C166EE, a standard size device, is implanted inside a portion of the aorta of a patient. The area E₁ in the in-use implantation model has been determined as presenting a high risk of endoleak. FIG. 10b represents a second example of in-use implantation model where a model of another Endurant II device, of type ETBF2816C166EE, is implanted inside the portion of the aorta of the same patient. The area E2 has been determined, with this other device, as presenting a low risk of endoleak.

1-13. (canceled)

14. A computer-implemented method for simulating an interaction between an endoprosthesis dedicated to be implanted in a portion of a patient aorta and said portion of said patient aorta, so as to determine areas with risk of surgical complications, said method comprising:
receiving 3D images of said portion of the patient aorta,
reconstructing a deformable tridimensional digital model of said portion of the patient aorta based on said 3D images, the reconstructed deformable tridimensional digital model being referred to as aorta model,
positioning a tridimensional digital model of said endoprosthesis at a planned location in said aorta model, said tridimensional model of said endoprosthesis and said aorta model forming together an implantation model,
simulating said interaction based on a computation using said implantation model,
determining, in the implantation model, based on results of said simulated interaction, at least one risk index of surgical complications for predefined areas,
providing as output said at least one risk index of surgical complications for predefined areas.

15. The computer-implemented method according to claim 14, wherein said surgical complications comprise at least one among: endoleaks, endoprosthesis migration, endoprosthesis infolding, endoprosthesis occlusion, thrombosis, and endoprosthesis kinks.

16. The computer-implemented method according to claim 14, wherein the computation involving said implantation model comprises a computation of a geometrical deformation of said tridimensional digital model of said endoprosthesis inside the aorta model.

17. The computer-implemented method according to claim 16, wherein the computation of the geometrical deformation is performed by finite element analysis.

18. The computer-implemented method according to claim 14, wherein said 3D images are preoperative CT scan images.

19. The computer-implemented method according to claim 14, wherein the interaction is an endovascular repair of aortic aneurysms.

20. The computer-implemented method according to claim 14, further comprising, after determining the areas with risk of surgical complication, determining a risk level for each area with risk of surgical complication.

21. The computer-implemented method according to claim 20, wherein determining a risk level comprises a computation involving an apposition index, representative of an apposition between the aorta model and said tridimensional digital model of said endoprosthesis, or an aspect ratio.

22. The computer-implemented method according to claim 20, wherein each risk level is associated with a corresponding display pattern, the method further comprising applying the corresponding display pattern to each area with risk of surgical complication.

23. The computer-implemented method according to claim 22, further comprising displaying the implantation model on a visualization unit with the areas with risk of surgical complication with the applied corresponding display pattern.

24. Method for planning an abdominal aortic aneurysm repair surgery using areas with risk of surgical complications determined by the computer-implemented method according to claim 14.

25. A device comprising a processor configured to carry out a computer-implemented method according to claim 14.

26. A non-transitory program storage device, readable by a computer, tangibly embodying a program of instructions executable by the computer to perform a computer-implemented method of claim 14.
